(19) United States
(12) Patent Application Publication
(10) Pub. No.: US 2004/0148000 A1
(43) Pub. Date: Jul. 29, 2004

(54) Self expanding stent delivery system with balloon

(76) Inventor: Fertac H. Bilge, Santa Rosa, CA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/350,820

(22) Filed: Jan. 24, 2003

(51) Int. Cl. A61F 2/06

(52) U.S. Cl. 623/1.11; 623/1.23

(57) ABSTRACT

A self-expanding stent delivery system and method is provided where the self-expanding stent is loaded in a catheter and is held in a collapsed position over a balloon. The stent is deployed and then the balloon may be used for post-dilitation. After the stent is released the balloon is substantially aligned in position for post-dilitation. In one embodiment, the balloon working length is substantially the same as the deployed stent length so that balloon dilitation outside the length of the stent and potential healthy tissue damage is minimized. In another embodiment, the balloon working length is shorter than the deployed stent length and the post dilitation is accomplished by positioning and inflating the balloon in a plurality of iterations.

### FIELD OF THE INVENTION

This invention relates to a self-expanding stent and delivery system where the self-expanding stent is loaded in a catheter and is held in a collapsed position over a balloon. The stent is deployed and the balloon may be used for post-dilitation.

### BACKGROUND OF THE INVENTION

Endoluminal prostheses have been used in a number of medical applications. They typically comprise medical devices that are adapted for temporary or permanent implantation within a body lumen, including both naturally occurring and artificially made lumens. Examples of lumens in which endoluminal prostheses may be implanted include, without limitation: arteries such as those located within coronary, mesentery, peripheral, or cerebral vasculature; veins; gastrointestinal tract; biliary tract; urethra; trachea; hepatic shunts; and fallopian tubes. Various types of endoluminal prostheses have been developed, each providing a uniquely beneficial structure to modify the mechanics of the targeted luminal wall. Frequently stents are used to prop open such body lumens.

Some of the endoluminal prostheses have been balloon expandable stents. Balloon catheters are used to deploy stents that are balloon expanded. In such systems, a stent is crimped on balloon on the tip of a catheter. The catheter is position in a body lumen with the stent adjacent a deployment site in the lumen. The balloon is then inflated to expand the stent into position in the lumen. In such systems, a restraining mechanism is not required to restrain the stent because the stent does not expand on its own, thus permitting a low profile stent.

Self-expanding endoluminal prostheses have been deployed in a number of applications such as, for example, in vascular stents for propping open vessels. These types of stents may be useful, for example, where the diameter of the body lumen varies along the length of the stent deployment site because they tend to expand to the diameter of the vessel. They may also be used in other indications. They have been used, for example, in indications for coronary, saphenous veins, peripheral, iliac, superficial femoral artery (SFA), superficial femoral, carotid, subclavian and biliary applications. Self-expanding stents are generally made of support structures such as, for example, rings of a spring-like material or a shape memory alloy that may be constrained in a radially compressed position for delivery in a catheter, and that tend to radially expand when released from the radially compressed position. The support structures exhibit sufficient radial force for the intended purpose of the device, e.g., to prop open vessels. Generally, self-expanding stents are deployed in a body lumen using a delivery catheter. A stent is loaded in a low profile catheter with a restraining mechanism holding the stent in a radially constrained position, for example with a sheath over the stent. The catheter is guided to the location where the stent is to be deployed and the sheath is retracted or the restraining mechanism is released, permitting the stent to expand into position in the body lumen.

In some instances, once the stent is deployed in the body lumen, a user will then insert a balloon catheter and further dilitate the stent and/or lesion site to ensure that the stent conforms to the body lumen wall and that it is fully opened. However, this requires introduction of a catheter and/or tracking of a balloon catheter within the body lumen. This additional tracking of a catheter into or through a body lumen increase the chance of additional damage to the vessels. This particular device has a disadvantage of potentially damaging healthy tissue proximal of the deployment site. A balloon on a catheter has been proposed for use in an abdominal aortic stent graft where the balloon is placed in a distal position prior to stent graft deployment. After deployment of the stent-graft, the catheter is tracked proximally until the balloon is located within the stent graft and is used to forcibly mold the spring portion of the stent graft into conforming fixed engagement with the interior surface of the vessel without causing inelastic deformation of the spring portion. However, the balloon is not pre-positioned with the stent-graft over it when the stent graft is deployed and when repositioned, the catheter tip is extended proximally into a region of healthy tissue. Furthermore, in situations where a balloon is be positioned into the stent, if the balloon is not precisely located adjacent the deployment site when the balloon is inflated, it may extend beyond the treated lesion into healthy tissue, possibly disrupting or damaging the healthy tissue. It would be desirable to avoid the possibility of disrupting or damaging healthy tissue adjacent a lesion site treated with a stent.

Accordingly, it would be desirable to provide a system for post dilitation of a self-expanding stent that reduces the possibility of damaging adjacent tissue. It is also desirable to provide a system for post dilitation of a self-expanding stent that does not require use of an additional balloon catheter, additional catheter tracking and/or substantial additional balloon catheter positioning.

### SUMMARY OF THE INVENTION

The present invention provides a self-expanding stent delivery system and method where the self-expanding stent is loaded in a catheter and is held in a collapsed position over a balloon. The stent is deployed and the balloon may be used for post-dilitation. After the stent is released the balloon is substantially or approximately aligned in position for post-dilitation. In one embodiment, the balloon working length is less than or equal to the deployed stent length so that balloon dilitation outside the length of the stent and potential healthy tissue damage is minimized. In another embodiment, the balloon working length is shorter than the deployed stent length and the post dilitation is accomplished by positioning and inflating the balloon in a plurality of iterations. By using a balloon shorter than the stent length for dilitating any vessel tapering may be accommodated as the balloon is deployed along the length of the stent.

### BRIEF DESCRIPTION OF THE DRAWINGS

FIG. 1 is a schematic view of a distal end of a stent delivery system according to one embodiment of the invention.

FIG. 2 is a schematic view of the proximal end of the stent delivery system of FIG. 1.

FIG. 3 is a schematic view of the delivery system of FIG. 1 after the stent has been deployed.

FIG. 4 is a schematic view of the delivery system of FIG. 1 in which the balloon is used for post-dilitation.

FIG. 5 is a schematic view of an alternative embodiment of the delivery system with a stent deployed in a body lumen.

### DETAILED DESCRIPTION OF THE DRAWINGS

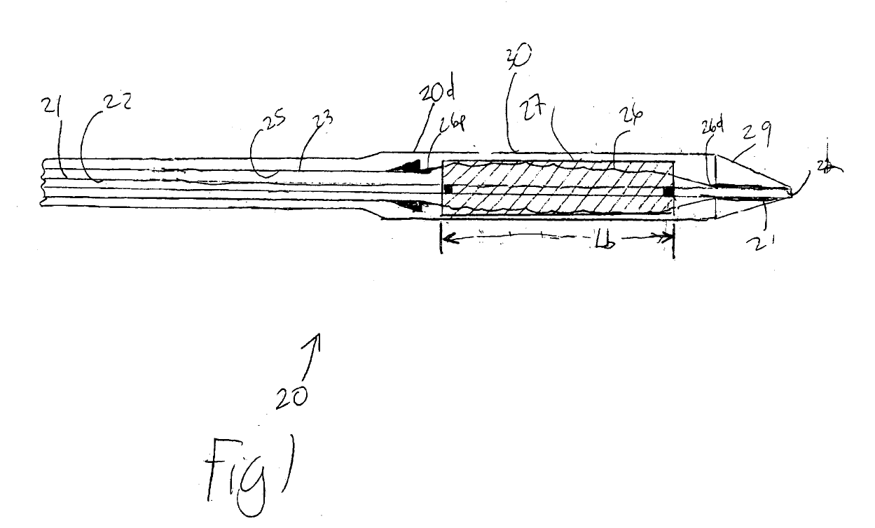

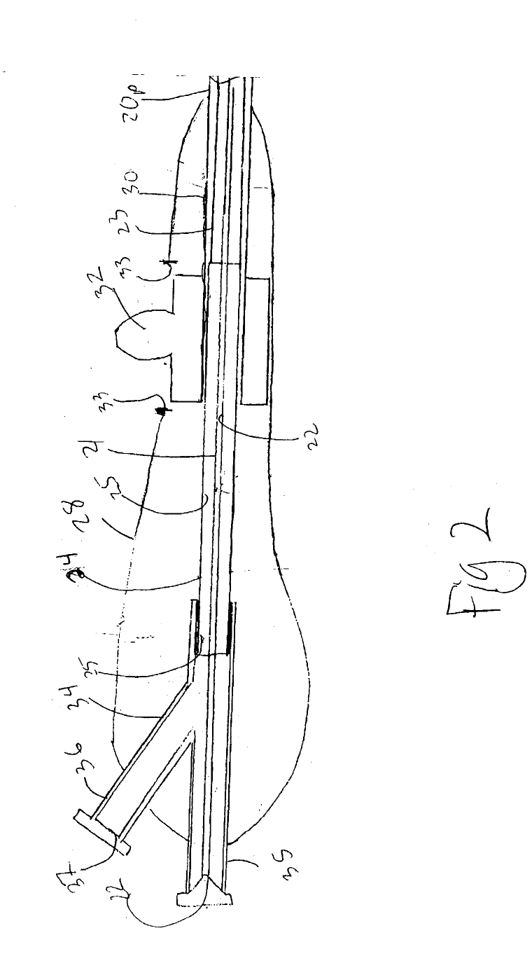

Referring to FIGS. 1-4, a stent delivery system according to one embodiment of the invention is illustrated. A catheter 20 comprises a proximal portion 20p having a handle 28 including a two-arm luer 34 and a distal portion 20d including a tapered distal tip 29. An inner tube 21 forming a guidewire lumen 22 extends from the proximal portion 20p to the distal portion 20d of the catheter 20. At the distal portion 20d, the tapered tip 29 is bonded over the distal end of the inner tube 21 so that the guidewire lumen 22 opens at the distal tip 29. The tapered distal tip 29 provides a smooth transition to the catheter 20 when the tip 29 is advanced through a body lumen, thus minimizing trauma to the tissue. At the proximal portion 20p, the inner tube 21 extends into a first luer 35 of two-arm luer 34 thus providing an opening into the guidewire lumen 22 from the handle 28. A guide wire may be inserted into the handle 28 from the two-arm luer 34 through the guide wire lumen 22, and may exit the guide wire lumen 22 through the tip 29 in the catheter 20.

An inflation tube 23 over the inner tube 21 forms a portion of an inflation lumen 25 concentric with the guidewire lumen 22 and extends from the proximal portion 20p to the distal portion 20d where it terminates just proximal of the distal end of the inner tube 21. A balloon 26 located at the distal portion 20d of the catheter 20 is bonded on its distal end 26d to the inner tube 21 and on its proximal end 26p to the inflation tube 23 where the inflation tube 23 terminates. The inflation tube 23 thus may supply an inflation medium through the inflation lumen 25 to inflate the balloon 26. The proximal end of the inflation tube 23 is coupled (e.g., bonded) to a hypotube 24, which extends into the handle 28 and is coupled (e.g. bonded) to the second luer 36 of the two-arm luer 34 to provide a continuous inflation lumen 25 through both the inflation tube 23 and the hypotube 24. The second luer 36 of the two-arm luer provides an inflation port 37 in fluid communication with the inflation lumen 25. Thus a source and pump for inflation medium to inflate and deflate the balloon 26 may be coupled to the inflation port 37.

The catheter 20 further comprises a sheath 30 slidably positioned over the inflation tube 23. The sheath 30 terminates at the handle 28 on the proximal portion 20p out of which an actuating button 32 coupled to the sheath 30 extends. The handle 28 includes a slit 33 configured to permit the button 32 to slide distally or proximally to advance or retract the sheath 30 respectively to or from a position in which it covers or exposes the balloon 26. When the catheter 20 is loaded, a stent 27 is compressed or rolled down to a small profile over the balloon 26, which is folded about the inner member 21. The stent 27 is a self-expanding stent and may be of a self-expanding stent design typical of those known in the art. The sheath 30 contains or constrains the stent in place over the balloon 26. In this embodiment, the stent length when deployed Ls, is substantially the same as the working length Lb of the balloon 26. The working length Lb of the balloon 26 is the portion of the balloon 26 that effectively may be used to dilitate the vessel or stent and accordingly would not include the tapered portion of the balloon that would not substantively dilitate the vessel or stent.

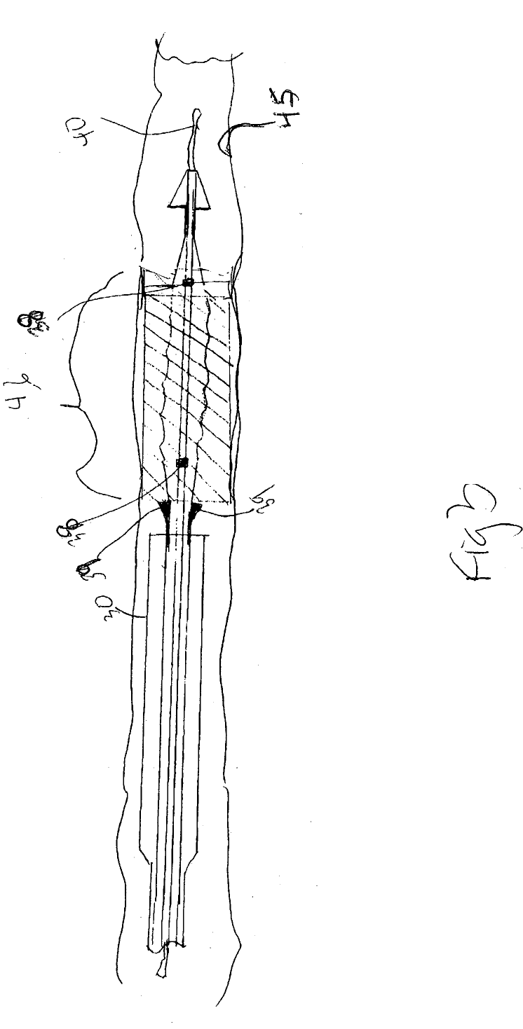

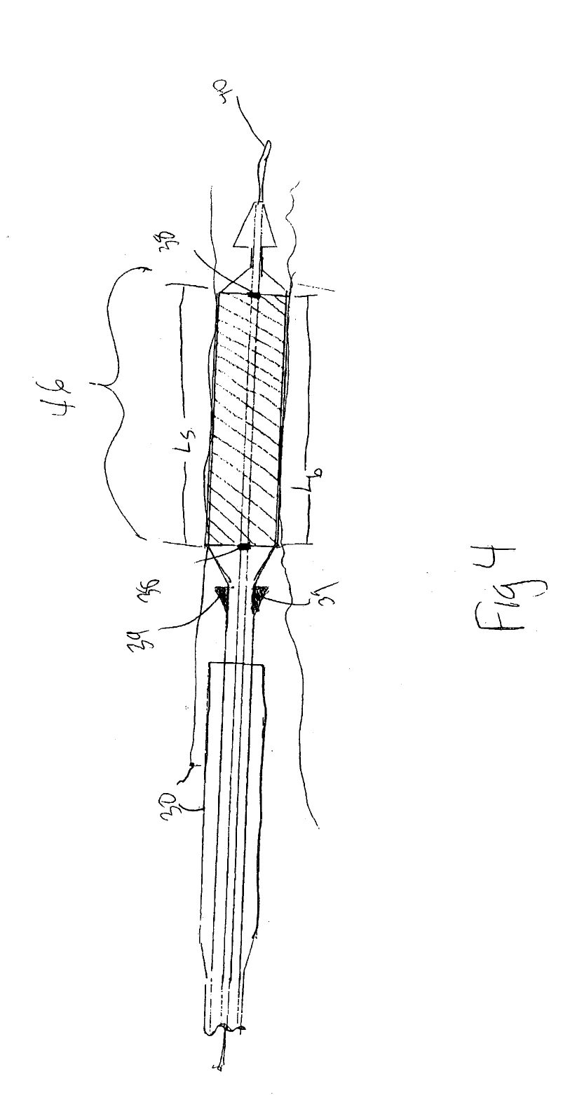

As illustrated in FIGS. 3 and 4, in use a guide wire 40 is positioned in a body lumen 45 adjacent a site 46 (such as a lesion) where the stent 27 is to be deployed. The catheter 20 loaded with the stent 27 is placed over the guide wire 40, which extends through the guidewire lumen 22. Using fluoroscopic imaging techniques, and aligning the stent 27, or markers 38 on the catheter 20 (inner member/inflation tube 23 under the balloon 26 that define the balloon working length and in this embodiment, the approximate stent length) within the deployment site 46. The sheath 30, which is restraining the stent 27, is retracted by moving the button 32 in a proximal direction to uncover the stent 27. Stent stop 39 comprises a tapered annular ring is bonded to and extends radially from the outer circumference of the inflation tube 23 just proximal of and adjacent the stent 27. The stent stop 39 guides the stent 27 when released, preventing the stent 27 from jumping back by the retracting movement of the sheath 30. Thus the stent stop 39 assists in maintaining substantial alignment between the stent 27 and the balloon 26 after the stent 27 is released. The stent 27 expands and engages the wall of the lumen 45 at the site 46. When the stent 27 is released, the balloon 26 is exposed and already in position adjacent the deployed stent 27 at the site 46. The balloon 26 may be repositioned so that its working length Lb is precisely aligned with the deployed length Ls of the stent 27. However, any such repositioning will be minimal. The balloon 26 may then be inflated to post-dilitate the site 46 and the stent 27 without requiring substantial repositioning of the catheter 20.

The balloon may be a compliant or semi-compliant balloon inflated to a given size at a given pressure. The balloon may be constructed for example of a nylon material such as Aved 100, pebax or PET. The balloon 26 may be inflated with contrast media so that its position is visible using fluoroscopic imaging.

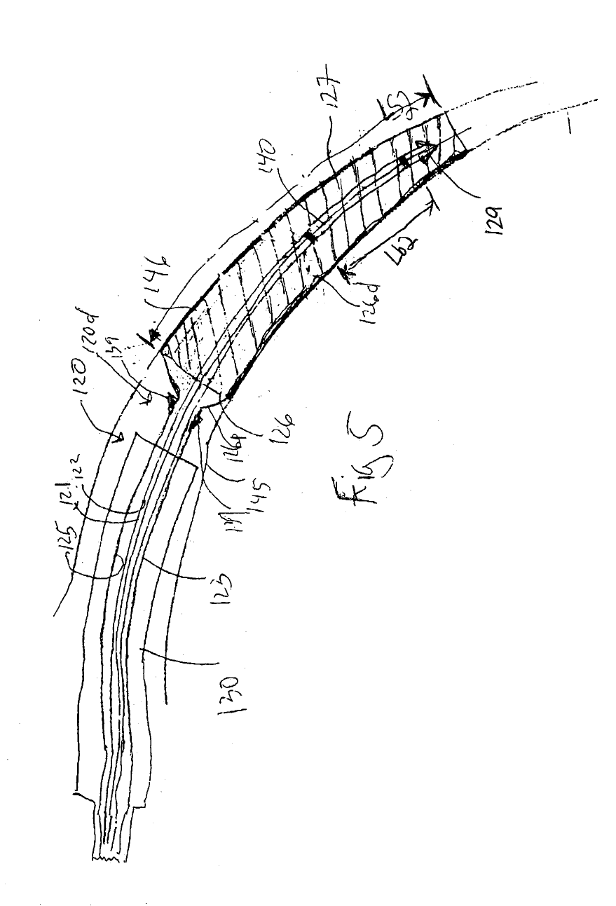

Referring to FIG. 5, an alternative embodiment of the invention is illustrated. A catheter 120 is illustrated in place in an artery 145 in which the anatomy is tapered and curved. The distal portion 120d of the catheter 120 comprises an inner tube 121 forming a guidewire lumen 122 through a tapered tip 129 at the distal end of the inner tube 121. The tapered distal tip 129 provides a smooth transition from the tip 129 to the diameter of the catheter 120 when the tip 129 is advanced through the artery 145, thus minimizing trauma to the tissue. The catheter 120 further comprises an inflation tube 123 over the inner tube 121 forming an inflation lumen 125 concentric with the guidewire lumen 122 and extending from the proximal portion (not shown) to the distal portion 120d where it terminates just proximal of the distal end of the inner tube 121. A balloon 126 located at the distal portion 120d of the catheter 120 is bonded on its distal end 126d to the inner tube 121 and on its proximal end 126p to the inflation tube 123 where the inflation tube 123 terminates. The inflation tube 123 thus may supply an inflation medium through the inflation lumen 125 to inflate the balloon 126.

The catheter 120 further comprises a sheath 130 slidably positioned over the inflation tube 123. The sheath 130 is actuable at the proximal end of the catheter 120 to retract the sheath 130 to release the stent 127 loaded in the catheter 120 and to expose the balloon 126.

As shown in FIG. 5, the catheter 120 has been placed over a guide wire 140 and has been positioned in an artery 145 adjacent a deployment site 146 (such as a lesion). The catheter 120 has been positioned with the balloon 126 and stent 127 aligned within the deployment site 146 and the sheath 130 has been retracted to release the stent 127. Stent stops 139 guide the stent 127 during release. The stent 127 as illustrated, has expanded and engages the wall of the lumen 145 at the site 146. The working length Lb2 of the balloon 126 is shorter that the deployed length Ls2 of the stent 126. The balloon 126 as shown in FIG. 5 is inflated to post-dilitate a portion of the stent 126. The balloon 126 is then to be positioned further along the stent 127 and dilitated again. This is repeated using fluoroscopic imaging to position the balloon within the stent 127 to dilitate the stent 127 along its length Ls2. The balloon 126 of this embodiment is a semi-compliant balloon so that it may be inflated to a size corresponding to the inner diameter of the artery at the location where the balloon is positioned by observation through imaging and/or by monitoring the balloon inflation pressure. Thus, the expanded diameter of the balloon may be varied along as the balloon is moved along the length of the stent to accommodate the inner diameter of the vessel.

Although this detailed description sets forth particular embodiments according to the invention, various other vascular grafts, endoluminal prostheses, and delivery systems are contemplated, especially those in which a self-expanding prosthesis is deployed.

While the invention has been described with reference to particular embodiments, it will be understood to one skilled in the art that variations and modifications may be made in form and detail without departing from the spirit and scope of the invention.

What is claimed is:

1. An endoluminal prosthesis delivery system comprising:
a delivery catheter comprising:
an elongate member having an inflatable member located on a distal portion of the elongate member and an inflation lumen extending through the elongate member and configured to deliver an inflation medium to the inflatable member to inflate the inflatable member;
an endoluminal prosthesis comprising:
a self-expanding tubular member having a first radially compressed configuration and a second expanded configuration, wherein the self-expanding tubular member is loaded in the catheter in the first radially compressed position over the inflatable member; and
a restraining member configured to restrain the self expanding tubular member in the first radially compressed configuration over the inflatable member, wherein the restraining member is actuable to release the self expanding tubular member to expand to the second expanded configuration, and wherein the inflatable member is configured to inflate inside the self expanding tubular member when in the second expanded configuration.

2. The endoluminal prosthesis delivery system of claim 1 wherein the inflatable member has a working length and wherein the working length of the inflatable member is substantially the same as an expanded length of the endoluminal prosthesis.

3. The endoluminal prosthesis delivery system of claim 1 wherein the inflatable member has a working length and wherein the working length of the inflatable member is less than an expanded length of the endoluminal prosthesis.

4. The endoluminal prosthesis delivery system of claim 1 wherein the endoluminal prosthesis has a deployed length when in the second expanded configuration, the delivery system further comprising:
a stent stop located on the delivery catheter adjacent the endoluminal prosthesis, wherein the stent stop is configured to guide the endoluminal prosthesis as it is being deployed to the second configuration so that when the endoluminal prosthesis is released from the restraining member, the inflatable member is substantially located within the deployed length of the endoluminal prosthesis.

5. The endoluminal prosthesis delivery system of claim 1 wherein the restraining member is a retractable sheath.

6. The endoluminal prosthesis delivery system of claim 1 wherein the inflatable member is a semi compliant balloon.

7. The endoluminal prosthesis delivery system of claim 1 wherein the inflatable member is capable of being deflated and re-inflated.

8. An endoluminal prosthesis delivery system comprising:
a delivery catheter means for delivering an endoluminal prosthesis to the inner circumference of a body lumen, the catheter means comprising:
an elongate means having an inflatable means for dilitating located on a distal portion of the elongate means and means for inflating the inflatable means;
an endoluminal prosthesis means comprising:
a self-expanding means having a first radially compressed configuration and a second expanded configuration, wherein the self-expanding means is loaded in the catheter means in the first radially compressed position over the inflatable means; and
a releasable restraining means for restraining the self expanding means in the first radially compressed configuration over the inflatable means, and releasing the self expanding means to expand to the second expanded configuration, and
wherein the inflatable means is configured to inflate within the self expanding means when in the second expanded configuration.

9. The endoluminal prosthesis delivery system of claim 8 wherein the inflatable means has a working length and wherein the working length of the inflatable means is less than or equal to an expanded length of the endoluminal prosthesis means.

10. The endoluminal prosthesis delivery system of claim 8 wherein the inflatable means has a working length and wherein the working length of the inflatable means is less than an expanded length of the endoluminal prosthesis means.

11. The endoluminal prosthesis delivery system of claim 8 wherein the inflatable means has a working length and wherein the working length of the inflatable means is substantially the same as an expanded length of the endoluminal prosthesis means.

12. The endoluminal prosthesis delivery system of claim 8 wherein the endoluminal prosthesis means has a deployed length when in the second expanded configuration, the delivery means further comprising:
a stent stop means for guiding the endoluminal prosthesis means during its deployment to the second configuration so that the inflatable means is substantially located within the deployed length of the endoluminal prosthesis means.

13. A method of deploying and endoluminal prosthesis comprising the steps of:
providing an endoluminal prosthesis delivery system comprising:
a delivery catheter comprising:
an elongate member having an inflatable member located on a distal portion of the elongate member and an inflation lumen extending through the elongate member and configured to deliver an inflation medium to the inflatable member to inflate the inflatable member;
an endoluminal prosthesis comprising:
a self-expanding tubular member having a first radially compressed configuration and a second expanded configuration, wherein the self-expanding tubular member is loaded in the catheter in the first radially compressed position over the inflatable member; and
a restraining member configured to restrain the self expanding tubular member in the first radially compressed configuration over the inflatable member, wherein the restraining member is actuable to release the self expanding tubular member to expand to the second expanded configuration, and wherein the inflatable member is configured to be inflated to dilitate the self expanding tubular member when in the second expanded configuration;
locating the distal portion of the catheter at a site for deployment of the endoluminal prosthesis;
releasing the endoluminal prosthesis from the restraining member; and
expanding the inflation member to secure the endoluminal prosthesis in place.

14. The method of claim 13 further comprising the steps of:
deflating the inflation member;
repositioning the distal portion of the catheter; and
re-inflating the inflatable member.

15. The method of claim 14 wherein the endoluminal prosthesis has a deployed length in the second expanded configuration; and
wherein the step of repositioning the distal portion of the catheter comprises repositioning the distal portion in a second location within the deployed length of the endoluminal prosthesis, wherein the diameter of the endoluminal prosthesis in the first location is different from the diameter of the endoluminal prosthesis in the second location.

16. The method of claim 13 wherein the step of expanding the inflation member to secure the endoluminal prosthesis in place comprises expanding the inflation member substantially within an expanded length of the endoluminal prosthesis.

17. The method of claim 13 wherein the inflation member has a working length and the endoluminal prosthesis has a deployed length when in the second expanded configuration; and
wherein the step of expanding the inflation member to secure the endoluminal prosthesis in place comprises expanding the inflation member so that the working length of the inflation member is substantially the same as an expanded length of the endoluminal prosthesis.

18. The method of claim 13 wherein the inflation member has a working length and the endoluminal prosthesis has a deployed length when in the second expanded configuration; and
wherein the step of expanding the inflation member to secure the endoluminal prosthesis in place comprises expanding the inflation member so that the working length of the inflation member is less than an expanded length of the endoluminal prosthesis.

19. The method of claim 18 wherein the step of positioning the distal portion of the catheter within a body lumen comprises positioning the catheter within a body lumen having a tapered diameter.

20. The method of claim 18 wherein the inflation member has a working length; and
wherein the step of expanding the inflation member to secure the expandable member in place comprises:
expanding the inflation member so that the working length of the inflation member is within an expanded length of the endoluminal prosthesis;
deflating the inflation member; and
re-inflating the inflation member so that the working length of the inflation member is within the expanded length of the endoluminal prosthesis.

21. The method of claim 13 wherein the step of expanding the inflatable member comprises expanding the inflatable member without substantial movement of the catheter after releasing the endoluminal prosthesis.
